(19) United States
(12) Patent Application Publication
(10) Pub. No.: US 2022/0339305 A1
(43) Pub. Date: Oct. 27, 2022

(54) SYSTEM FOR DISINFECTING OBJECTS

(71) Applicant: Thomas Ellis Stanley Haskins, Holbrook, MA (US)

(72) Inventor: Thomas Ellis Stanley Haskins, Holbrook, MA (US)

(21) Appl. No.: 17/239,658

(22) Filed: Apr. 25, 2021

(51) Int. Cl. A61L 2/10; A61L 2/24; A61L 2/26

(52) U.S. Cl. CPC A61L 2/10; A61L 2/24; A61L 2/26; A61L 2202/14; A61L 2202/11

(57) ABSTRACT

Disclosed is a system for disinfecting objects in an environment. The system includes a UV light source for disinfecting the objects in an environment, an attachment unit to attach the UV light source to a fixed support, and a switch to operate the UV light source. The switch is also an automatic timer programmed to perform for a pre-defined duration. Further, the system includes a cabinet for housing the UV light source, a sensor to automatically shut down the UV light when door of the cabinet opens and a platform to rotate the objects inside the cabinet. Further, the system may include a conveyor belt to carry the objects under the UV light.

### BACKGROUND OF THE INVENTION

### 1. Field of the Invention

The present invention relates to disinfecting objects, and with particular attention to a system for disinfecting objects used regularly in daily life by the average consumer, whether at work, home or out and about.

### 2. Description of Related Art

It is well known in the art to use ultraviolet light (UV) for the disinfection treatment of water. Ultraviolet light, at the germicidal wavelength of 253.7 nanometers, alters the genetic (DNA) material in cells so that bacteria, viruses, molds, algae and other microorganisms can no longer reproduce. The microorganisms are considered dead, and the risk of disease from them is eliminated.

Ultraviolet light is classified into three wavelength ranges: UV-C, from 200 nanometers (nm) to 280 nm; UV-B, from 280 nm to 315 nm; and UV-A, from 315 nm to 400 nm. Generally, UV light, and in particular, UV-C light is “germicidal,” i.e., it deactivates the DNA of bacteria, viruses and other pathogens and thus destroys their ability to multiply and cause disease, effectively resulting in sterilization of the microorganisms.

Specifically, UV “C” light causes damage to the nucleic acid of microorganisms by forming covalent bonds between certain adjacent bases in the DNA. The formation of these bonds prevents the DNA from being “unzipped” for replication, and the organism is unable to reproduce. In fact, when the organism tries to replicate, it dies. UV light with a wavelength of approximately between about 250 to about 260 nm provides the highest germicidal effectiveness.

Various UV devices have been proposed to reduce infectious pathogens. For example, bathrooms in airplanes have started to use UV LED strips to reduce pathogens while in flight. Other facilities are being outfitted with various devices to expose pathogens to UV light sources. However, UV light sources have generally been independent or standalone devices that are specifically designed for intermittent applications.

An example is the use of UV light in barber shops for the sterilization of combs and cutting instruments. Ultraviolet light may be produced artificially by electric-arc lamps. Ultraviolet light may also be generated by mercury bulbs. Such light sources have been used for disinfection and sterilization of objects.

Accordingly, there are various methods to reduce microbial infections. However, specifically in health-care environments, it is not enough to reduce the microbial population to a certain amount; the microbial population has to be eliminated. Even the smallest amounts of microbes in health-care environments may be detrimental. UVC light is the most effective form for this purpose.

Moreover, methods such as UV light cannot be used in every situation given that it does not penetrate rather it affects only the surface. Moreover, UV is toxic for human tissues and eyes and cannot be used everywhere and all the time. Therefore, there is a need of a safe, non-toxic system that disinfects and sterilize objects in an environment.

### BRIEF DESCRIPTION OF DRAWINGS

The devices of the invention each have several aspects, no single one of which is solely responsible for its desirable attributes. Without limiting the scope of this invention as expressed by the claims which follow, its more prominent features will now be discussed briefly. After considering this discussion, and particularly after reading the section entitled “Detailed Description of the Embodiments of the Invention” one will understand how the features of this invention provide advantages.

FIG. 1 illustrates a schematic view of system for disinfecting objects; and

FIG. 2 illustrates another schematic view of the system for disinfecting objects inside a cabinet. The version in the cabinet would be controlled by the door switch and the timer which would be on the outside of the cabinet.

FIG. 4 illustrates another schematic view of a system for disinfecting objects such as dish scrubbers and dish rags by providing an enclosed and shielded contain with a plurality of compartments riding on a half “lazy susan” device that can be turned to bring each or all the compartments out of the enclosed device to remove or add objects to be disinfected, whereas inside the device is a UVC light controlled by a timer and a light switch activated by the lazy susan being closed.

FIG. 4 illustrates a side view of the lazy susan disinfecting device.

FIG. 5 illustrates a schematic view of a version of this device designed specifically to disinfect dish rags and anything that can be hung on a rung and that will fit in the device. This version does not have a lazy susan but rather a front door.

FIG. 6 illustrates a schematic view of a version of a this device designed to hold and disinfect cooking utensils while user is making something to eat.

FIG. 7 illustrates a schematic side view of FIG. 6.

### DETAILED ITEMIZATION OF OBJECTS BEING REFERENCED

100 System
102 UV light source
104 Attachment unit
106 Light switch
202 objects to be disinfected
204 cabinet
206 controller
208 door
210 door switch
212 platform
214 motor
300. Batteries
301. Battery cabinet
302. Control unit
303. Electrical wires
304. Switch trigger
305. Face of unit
306. Inner partitions
307. Rotation handle
308. Frame wall
400. Rotation handle pivot support/attachment shaft
401. Closed top section
402. Rotation handle and lazy susan pivot base
403. Switch arm guide
404. Switch off stop
405. Switch off stop
406. Sending electrical wire
407. switch on stop
408. switch trigger
409. Switch arm end roller
410. switch arm
411. lazy susan
412. enclosed container
500. door hinges
501. doorknob
502. hanging rungs
503. hanging rags
600. roll of plastic wrap
601. UV light cover hinge
602. UVC light cover
603. cutting edge
604. cutting edge holder
605. plastic wrap end
606. utensils
607. Utensil placeholders
608. Roll holder
609. Roll rollers
610. Cutting edge holder attachment brackets
611. Utensil placeholders depressions
612. Plastic wrap
613. Unit platform

### DETAILED DESCRIPTION OF DRAWINGS

Embodiments of the invention will now be described with reference to the accompanying Figures, wherein like numerals refer to like elements throughout. The terminology used in the description presented herein is intended to be interpreted in its broadest reasonable manner in accordance with its ordinary use in the art and in accordance with any overt definitions provided below.

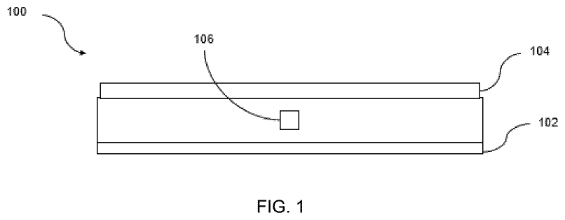

FIG. 1 illustrates a schematic view of a system 100 for disinfecting objects. The system 100 includes a UV light source 102 for disinfecting the objects in an environment, an attachment unit 104 to attach the UV light source 102 to a fixed support, and a light switch 106 to operate the UV light source 102. Examples of the UV light source 102 include but not limited to black lights, curing lamps, germicidal lamps, mercury vapor lamps, halogen lights, high-intensity discharge lamps, fluorescent and incandescent sources, and some types of lasers having wavelength between 250 to about 260 nm etc.

Examples of the attachment unit 104 include but not limited to adhesives, double side adhesives, screw holes with screws, bolt holes with bolts and other similar attachment units. Examples of the fixed support includes but not limited to walls, floors, roofs, and other similar fixed areas in an environment. Examples of the environment include but not limited to a cabinet, box, retail shops, grocery stores, retail markets, shopping malls, a home or kitchen/bathroom, freezer/fridge, any area with a defined boundary etc.

The switch 106 is designed (via small computer chip) to light only long enough after closing door to disinfect the area covered. The switch 106 is a timer switch to automatically start operation when the door is closed and automatically offs the operation when the door is opened. For exemplary purposes, the switch 106 operates for a pre-defined period such as but not limited to 15 minutes, 30 minutes, 60 minutes etc.

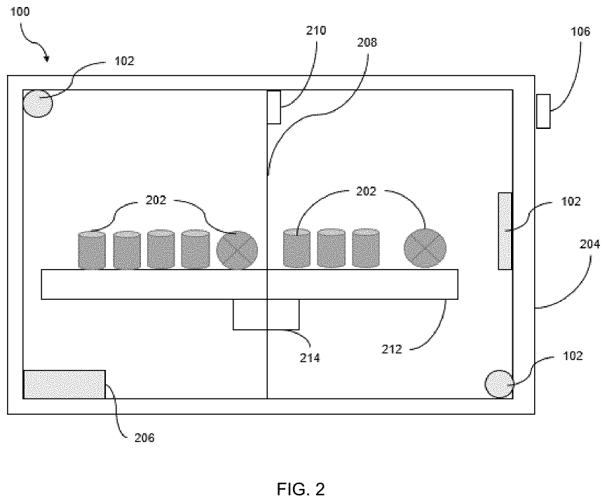

FIG. 2 illustrates another schematic view of the system 100 for disinfecting objects 202 inside a cabinet 204. The cabinet 204 houses the UV light source 102 and the attachment unit (not shown in FIG. 2) and the switch 106 is attached to the cabinet 204. The system 100 includes a controller 206 to automatically shut down the UV light source 102 when door 208 of the cabinet 204 opens, a door switch 210 operated by the controller 206, a rotating platform 212 to rotate the objects 202 inside the cabinet 204 and a motor 214 to rotate the rotating platform.

Examples of the controller 206 include but not limited to an electronic timer, pressure sensor (this pressure sensor is 210) etc. Examples of the door switch 210 include but not limited to a mechanical switch or a magnetically activated switch. Examples of the cabinet 204 include but not limited to refrigerators, freezers, chambers, cabinets for food, medicines, cabinets with shelf, and other similar enclosed environments.

Examples of the platform 212 include but not limited to a Lazy Susan, mechanical belts and other similar items for placing objects. Examples of motor 214 include but not limited to DC motors, brushless DC motor etc. for rotating the belt 212. Examples of the object 202 include but not limited to utensils, groceries, crockery, goods, clothes, combs and brushes, medical objects, packets etc.

The controller 206 automatically controls the motor 214 and the UV light source 102, when door 208 is closed. The controller 206 automatically energizes blue lights and operates lazy susan long enough and at the optimal speed (platform) to disinfect all surfaces of content objects 202. Further in another embodiment of the present invention, the system 100 may include a conveyor belt to carry the objects under the UV light, travelling from the cabinet entrance to exit. Yet another in another embodiment of the present invention, the system 100 may include a cabinet designed to fit over and existing conveyor belt such as in a grocery store checkout area to bath the objects with the UV light as they travel along the existing conveyor belt. The conveyor belt is used in big retails, shopping complexes, groceries etc.

In alternative embodiment of the present invention, the system 100 further includes plurality of mirrors. At least one mirror is attached to inside walls of the cabinet 204 for reflecting the UV light. The mirrors allow for the need of fewer UV light sources 102.

For exemplary purposes, place groceries (etc.) inside unit and close doors. Once doors are closed the UV light turns on and disinfects all surfaces. Automatic timer chimes when enough time has elapsed. UV light covers from 3 opposing corners (if mirrors not installed) as lazy susan rotates contents. This causes the light to disinfect all sides, top and bottom of contents as long as contents do not block light from each other. An alternate version is portable and battery powered and lazy susan can also be operated by battery or in a less expensive and lighter portable version the lazy susan can be operated by a hand crank. This portable version could have solar energy cells to recharge batteries. Another portable version will have the lazy susan and the bluelight all operated by a hand crank. In this case the hand crank manually rotates the lazy susan and also generates electricity to energize the blue lights (without the need for any batteries).

FIG. 3 illustrates another schematic view of the system 100 and is generally identical with the following differences:

This embodiment of the invention is significantly smaller and designed to receive smaller items and in fewer quantities such as dish scrubbers and having a plurality of independent, smaller compartments built into the lazy susan 411 to hold such items separately under the UV light 102 for disinfecting. This embodiment includes all the same items as those in FIG. 1 except for the door 208 or doors 208 and this embodiment illustrates a battery 300-powered version, however, it could also be powered in others ways such as, but not limited to, solar, direct building current, spring tension (like a music box). In this illustration the lazy susan 411 is operated to turn the inner partitions out of the enclosed cabinet 412 for user access to add or remove objects. The process of operating the lazy susan 411 in this illustration is achieved by turning the rotation handle 307 which is attached to the lazy susan 411, however, an alternative version of this embodiment could be equipped with an electrical switch and a rotation motor. When the lazy susan 411 is turned so that one or more of its compartments is exposed for access by user, the light switch will turn to the off position so that the user's eyes and skin are not harmed by the light. When the lazy susan 411 is turned so that all its inner partitions 306 are inside the enclosed device 412 the light switch 106 will return to the on position and the timer in the control unit 302 will allow the light to stay on for 30 second. An alternative version of this embodiment can have multiple options of time duration for the light to be left on for disinfection. This illustration shows the batteries 300, battery compartment 301, wires leading from batter compartment to control center and to the light switch 303. It also illustrates the face of the unit 305, switch trigger 304, inner partitions 306, the unit's frame wall 308 and the UVC light 106.

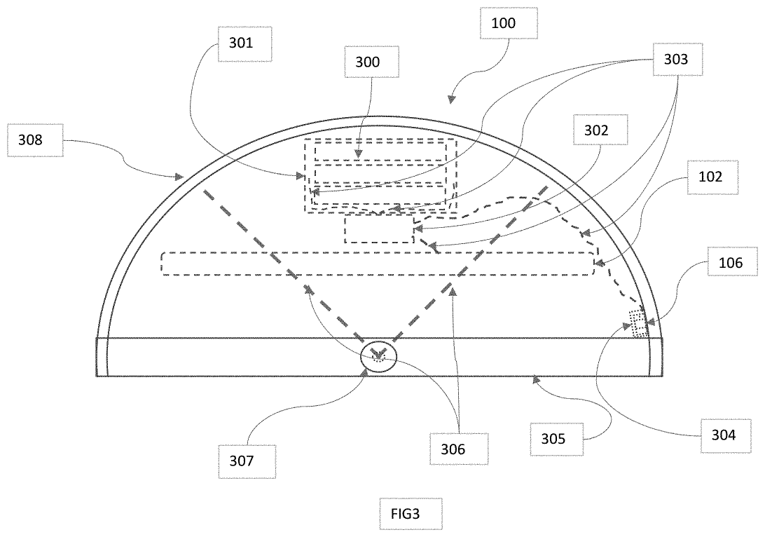

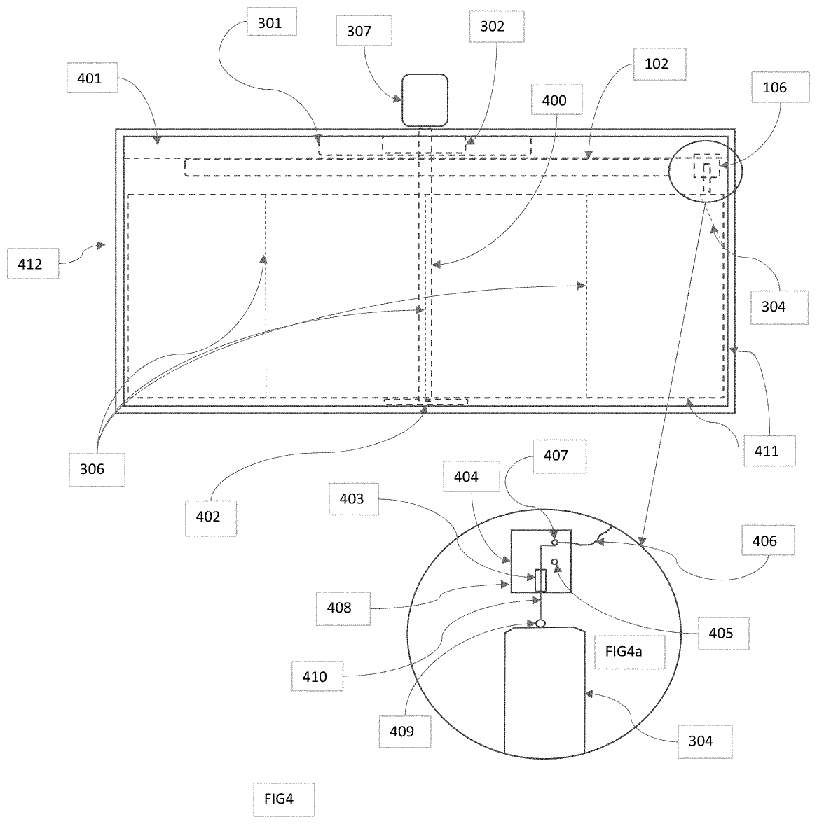

FIG. 4 illustrates another schematic is side view of FIG. 3 illustrating, in addition to what is illustrated in FIG. 3, the Rotation handle pivot support/attachment shaft 400, the rotation handle and lazy susan pivot base 402 and the detail of a possible embodiment of the light switch for this embodiment of the invention including the, the closed top section 401, the rotation handle and lazy susan pivot base 402, the rotation handle 307 is attached to the rotation handle pivot support/attachment shaft 400 which is attached to the rotation handle and lazy susan pivot base 402 which is attached to the lazy susan 411. When the rotation handle 307, therefore is rotated, the lazy susan 411 will rotate causing the inner partitions 306 to rotate in and out of the enclosed container 412.

FIG. 4a illustrates a schematic detailed view of one possible embodiment of the light switch 106 and its placement, mounting and triggering mechanism shown in FIG. 4 illustrating the switch arm pivot 403, the Switch off stop 404, the Switch off stop 405, the sending electrical wire 406, the switch on stop 407, the switch trigger 408, the Switch arm end roller 409, and switch arm 410. The illustration shows how the light switch 106 is moved to the on position only while the switch trigger 304 is placed directly under it and this only happens when the entire lazy susan 411 is rotated inside the enclosed container 412, thus allowing the UV light to shine only when it cannot escape to harm the user. When the switch trigger 304 pushes the switch arm 410 up through the switch arm guide 403 it moves it from the off position 405 to the on position 407. When the switch arm 410 comes in contact with the switch trigger 304 the switch arm end roller 409 protects the switch arm 410 from be bent by rolling onto the top surface of the switch trigger 304.

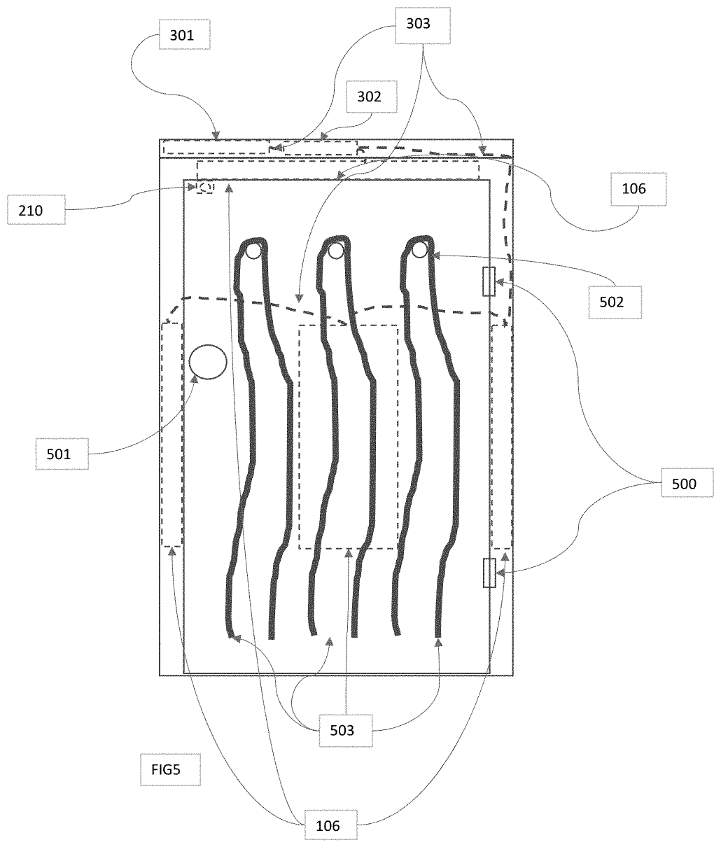

FIG. 5 illustrates another schematic view of the system 100 and is generally identical with the following differences:

The unit has one door 208 and has hanging rungs 502 on which to hang items to be disinfected such as rags 503. This unit is design specifically for disinfecting dish rags 503 and other cloth items, however, it can be used for disinfecting many other types of items. It has a plurality of UV lights 106, door hinges 500, a doorknob 501, a plurality of hanging rungs 502, a door light switch 210, a control unit 302, a battery cabinet 301, batteries 300 (however, an alternative embodiment of this unit can be powered by other methods, but not limited to, those mentioned in FIG. 2 and FIG. 3), electrical wires 303. To utilize this unit the door 208 is opened and a rag 503 or other item is hung on one of the hanging knobs 502, the door 208 is closed and this triggers the light switch 106 which allows the control unit 302 to turn on the UV light 102 for the desired time. The control unit 302 can also be set to turn the UV light 106 on periodically when door 208 is not opened frequently to maintain a disinfected environment and contents.

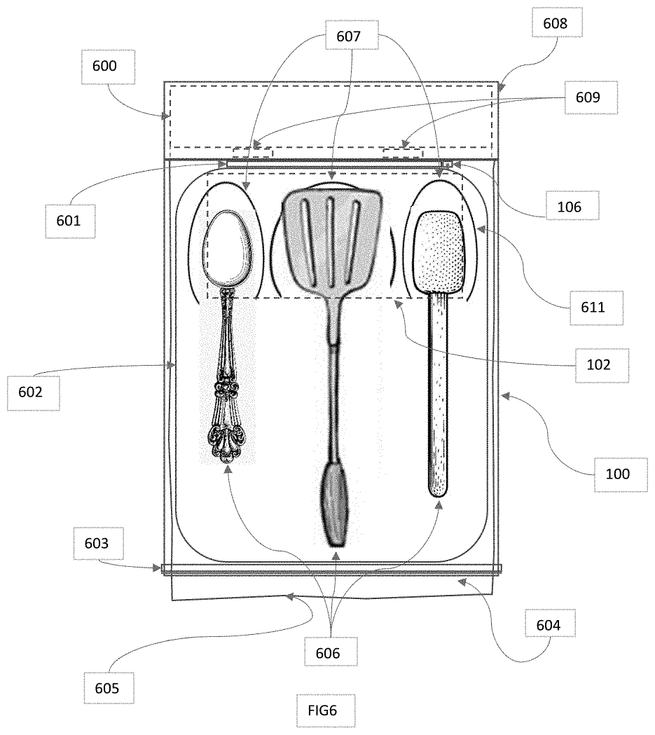

FIG. 6 illustrates a schematic view of another embodiment of the system 100 designed to lay flat on a surface such as a kitchen countertop and have cooking or other utensils 606 placed on it between frequent uses, having a roll of plastic wrap 600, such as is used to store food in topless containers, that is placed in the roll holder 608 and drawn out from there to cover the top of the unit platform 613 and drawn through the cutting edge holder 603 and under the cutting edge 604. When a given length of the plastic wrap 612 has been used the plastic wrap end 605 is then pulled further out of the roll holder 608 until the top end of the used portion slides past the cutting edge 603 at the end of the unit and then pulled up against the cutting edge 603 and torn away so that it may be disposed of. Ideally the top of the unit platform 613 will have one or more depressions to receive and hold in place the utensils 606 as they rest on top of the plastic wrap 612. Inside the roll holder 608 are a plurality of roll rollers 609 that help the roll of plastic wrap 600 to roll more easily inside the roll holder 608 while the plastic wrap 612 is being pulled out of the roll holder 608. Over all this can be rested a UV light cover 602 with an attached UV light 102 and which swinges up and down on one or more UV light cover hinges 601. This UV light cover covers the utensils 606 on the top of the unit platform 613 and prevents any UV light from escaping to harm the eyes or skin of the user. The UV light switch 106 turns the UV light 102 on only when the UV light cover 602 is placed fully down in covering position and the control unit 302 keeps the UV light 102 lit for 30 seconds or any other desired time.

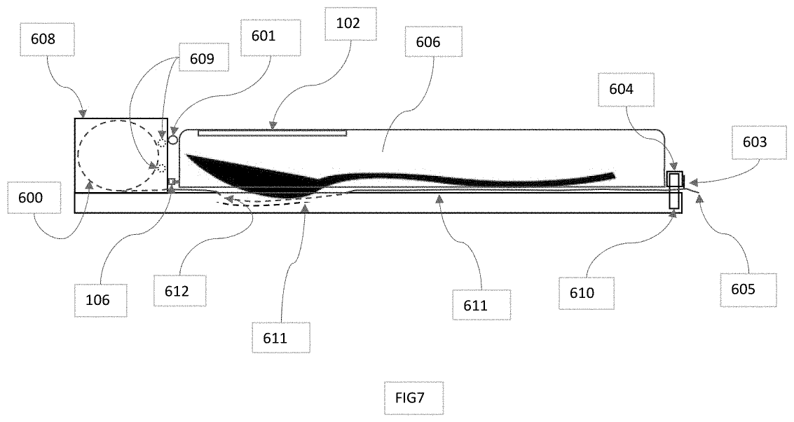

FIG. 7 illustrates a schematic of a side view of FIG. 6 with all the same items and characteristics illustrated plus the tendency of the plastic wrap 312 to sink into the utensil placeholder depressions 611 and the cutting edge holder attachment brackets 610 that adhere the cutting edge holder 604 to the end of the top of the unit platform 613, there being a gap between the cutting edge holder 604 and the top of the unit platform 613 and between the cutting edge 603 and the top of the unit platform 613, through both of which the plastic wrap 612 can be slid.

While the above detailed description has shown, described, and pointed out novel features of the invention as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all of the features and benefits set forth herein, as some features may be used or practiced separately from others. The scope of the invention is indicated by the appended claims rather than by the foregoing description. All changes which come within the meaning and range of equivalency of the claims are to be embraced within their scope.

1. A system for disinfecting objects in an environment, the system comprising:
a UV light source for disinfecting the objects in a closed an environment;
an attachment unit to attach the UV light source to a fixed support; and
a switch to operate the UV light source, and;
A control unit to control the time duration and timing of the operation of the UV light source, and;
comprising a plurality of mirrors, wherein at least one mirror attached to inside walls of the cabinet for reflecting the UV light.

2. The system according to claim 1 further comprising:
a cabinet for housing the UV light source and with or without a conveyor belt or a rotating platform to carry the objects under the UV light;
a controller to automatically shut down the UV light source when door or cover of the
cabinet opens, or in an alternative embodiment, and;
a switch operated which energizes the controller;
a platform to rotate the objects inside the cabinet, or in and out of the cabinet; and
a motor or mechanical method to rotate the platform.

3. A system with a platform with placement depressions to hold utensils, an attached plastic wrap roll holder to receive a roll of plastic wrap roll, an attached cutting edge to cut the plastic wrap, plastic warp roll rollers to help the plastic wrap roll more easily as the plastic wrap is pulled out and placed on top of the platform to keep the platform free of debris and liquids from the utensils, a UV light to disinfect the utensils, UV light switch attached to the plastic wrap roll holder and triggered by the UV light cover, a UV light cover to enclose the light and UV light cover hinges on which the UV light cover swings to open and closed positions.
